(19) United States
(12) Patent Application Publication
(10) Pub. No.: US 2024/0268915 A1
(43) Pub. Date: Aug. 15, 2024

(54) Instrument Guide with Angled Sensor

(71) Applicant: Shane S. Pak, Arcadia, CA (US)

(72) Inventor: Shane S. Pak, Arcadia, CA (US)

(21) Appl. No.: 18/436,988

(22) Filed: Feb. 8, 2024

Related U.S. Application Data
(60) Provisional application No. 63/445,265, filed on Feb. 13, 2023.

(51) Int. Cl. A61B 90/00

(52) U.S. Cl. CPC A61B 90/06; A61B 90/08; A61B 2090/067; A61B 2090/0811

(57) ABSTRACT

An instrument guide for use with a surgical or other instrument that positions an angular measurement sensor at a perpendicular angle to the shaft of the instrument. In this manner, where the sensor is programmed to work relative to gravity, or a vertical plumb line, the perpendicular placement allows the measurement and display of lateral angles relative to horizontal.

### CROSS-REFERENCE TO RELATED APPLICATION

This application claims the benefit of U.S. Provisional Application No. 63/445,265, filed Feb. 13, 2023, the contents of which are incorporated herein by reference.

### FIELD OF THE INVENTION

The field of the invention regards the alignment of instruments, including the angular alignment of surgical instruments such as those used in spinal stabilization surgeries, including laterally performed spinal surgery.

### BACKGROUND OF THE INVENTION

Thousands of spinal stabilization surgeries are performed every year. These surgeries typically involve tools, such as drills, awls and screwdrivers, to install stabilizing structures such as spinal rods and/or plates, which may be secure by pedicle screws. The implantation of pedicle screws may involve first drilling a pilot hole through the pedicle and into the vertebral body of the patient's spine. The pedicle screw may then be implanted into the pilot hole. In order to properly create the pilot hole and place the pedicle screw, and avoid damaging the patient's spinal column, the position, angular orientation and trajectory of the pilot hole must be precisely executed. This requires that the tools used in this process also be accurately positioned during use so that their angular orientation and trajectory are appropriate. If they are inaccurate, the pilot hole and/or screw could breach and/or perforate a cortical wall, thereby causing significant neurological injury.

To facilitate the accurate placement, angular orientation and trajectory of surgical instruments as they are used during surgery, innovative instrument alignment feedback systems and methods have been described in U.S. Pat. No. 11,484,381 (“'381 patent”), the disclosure of which is incorporated by reference as if fully set forth herein. An embodiment of an alignment feedback system disclosed in the '381 patent includes a sensor chip having an accelerometer that is oriented with the direction of gravity.

As explained in the '381 patent, this sensor chip may be attached to the surgical instrument so that the accelerometer, which is oriented with the direction of gravity, is also oriented along the axial direction of the surgical instrument. In other words, the accelerometer is generally aligned along the axial direction of the shaft of the surgical instrument. In this manner, the sensor chip may provide angular orientation of the surgical instrument in the sagittal and axial planes. Because the accelerometer in this embodiment is oriented with the direction of gravity, these angular orientation measurements may be provided with respect to a vertical plane or vertical plumb line.

The alignment feedback system embodiment described above is preferably used where it is desired to measure the angular orientation of the surgical instrument with respect to a vertical plumb line. This may generally be because the accelerometer used by the sensor chip is oriented along the direction of gravity.

However, there are increasing types of spinal surgery performed in the lateral position where the patient is positioned on his or her side, thereby positioning the spine laterally. In this situation, surgical procedures may be performed where the surgical instrument enters the patient laterally, i.e., so that the surgical instrument axis is still generally oriented at or near vertical. However, where the patient is lying on his or her side, the pedicles are oriented laterally, i.e., 90 degrees from vertical. For example, lumbar pedicle screws are generally implanted in the lateral position. SI joint fusions are also addressed from a lateral approach percutaneously. In these laterally oriented surgical procedures, the alignment and trajectory of surgical instruments remains very important to avoid neurological injury. However, the axis of a surgical instrument in these situations would be engaging the patient at, or close to, horizontal, i.e., at a significant angle from a vertical plumb line. This may create issues where the sensor chip is aligned with the axis of the surgical instrument and the sensor chip is programmed or otherwise designed to provide optimum accuracy for angular measurements with respect to a vertical plumb line.

However, in these lateral applications, if the sensor chip described above is attached to the surgical instrument so that its accelerometer is oriented along the shaft's axis, the accelerometer will generally be oriented at or near 90 degrees from the direction of gravity; or at a significant angle from a vertical plumb line. This may create issues where the sensor chip is programmed or otherwise designed to provide optimum accuracy for angular measurements with respect to a vertical plumb line.

Laterally performed spinal surgery procedures, where the patient is in a prone position, are being developed. In this situation, where the surgical instrument enters the patient laterally, the axis of the surgical instrument would again generally perpendicular or at a significant angle from a vertical plumb line. This may again create issues where the sensor chip is aligned with the axis of the surgical instrument and the sensor chip is programmed or otherwise designed to provide optimum accuracy for angular measurements with respect to a vertical plumb line.

Accordingly, there is a need for an alignment system and method that may accommodate surgeries in the lateral position, e.g., where the axis or shaft of the surgical instrument is generally positioned in lateral positions and/or at or near a horizontal plane.

There is also a need for an alignment system and method that may accommodate the use of surgical instruments in lateral positions, but that may still include sensors that are programmed or otherwise designed to be provide angular orientation with respect to a vertical plumb line, even though angular orientation information with respect to the floor or to a horizontal plane is desired.

For surgical procedures where surgical instruments may engage the patient with respect to a vertical plumb line, as well as with respect to horizontal, there is also a need for a set of surgical instruments whose axes and trajectories may be accurately displayed to the surgeon with respect to both vertical and horizontal.

### SUMMARY OF THE INVENTION

The present invention is specified in the claims as well as in the below description.

In an aspect of the invention, a lateral instrument with sensor system is described. The system may include a handle or other tool that may be held or manipulated by a surgeon, and that is configured to receive a surgical instrument, where the surgical instrument is generally held at a lateral position, e.g., on or around a horizontal plane. The handle or other tool may also be configured to receive a sensor that may provide information on the angular orientation of the surgical instrument. The sensor may include an accelerometer or other device that may provide such angular orientation information. The surgical instrument may be a drill or other types of surgical instruments, including those instruments having a shaft. The system may be configured so that the surgical instrument and the sensor are positioned at or about perpendicular to each other, i.e., the axis of the surgical instrument may be positioned on one plane, and the sensor may be positioned on another plane. For example, when the surgical instrument is positioned on or near a horizontal plane, the sensor is positioned on or near a vertical plane.

Another aspect of the invention regards the ability for the lateral system to use a sensor that is generally programmed to work with the direction of gravity. For example, the sensor that provides angular orientation of the surgical instrument may be designed to work when it is oriented with the force of gravity; or in other words, designed to work in more of a vertical orientation. In these situations, the sensor may not provide accurate angular orientation information if it is oriented in more of a horizontal, or lateral, position. In this aspect of the invention, the sensor may be positioned so that it is more in a vertical orientation while the surgical instrument is positioned in more of a horizontal or lateral orientation.

Another aspect of the invention regards the manner in which the tool or other mechanism described above may receive the surgical instrument. For example, the tool or other mechanism may include a hole or bore with an inner diameter, into which a surgical instrument may be inserted; where the outer diameter of the surgical instrument may correspond with the inner diameter of the hole or bore. As another example, the hole may have shape that corresponds to the shape of the surgical instrument. The surgical instrument may be secured to the tool by a friction fit. Alternatively, a locking mechanism may be included to secure the surgical instrument to the tool, such as a quick release lock.

Another aspect of the invention regards the manner in which the tool or other mechanism described above may receive the sensor. For example the tool or other mechanism may include a slot, bracket or other device to receive the sensor. As another example, the sensor may be integrally formed as part of the tool.

In another aspect of the invention, the surgical instrument itself may be configured to receive a sensor. For example, an awl, screwdriver or cannulated needle may be configured to receive a sensor that provides information on the angular orientation of such surgical instruments. Other types of surgical instruments, such as instruments having a shaft, may be included in this aspect of the invention. The sensor, which may be programmed to provide angular measurements with respect to a vertical plumb line, may be attached to the surgical instrument so that it is generally positioned to be perpendicular to the shaft of the instrument. In this manner, when the surgical instrument is used in a lateral position, i.e., at or near horizontal, the sensor will be generally aligned at or near vertical. As such, the sensor's angular measurements with respect to vertical may equate to the instrument's angular measurements with respect to horizontal, i.e., lateral.

Another aspect of the invention regards the foregoing but used with non-surgical instruments and tools, i.e., any instrument, tool or other device, the use of which, and accuracy of such use, may benefit from lateral angular measurements provided by the current invention.

Another aspect of the invention regards a system including the lateral tool with the vertically oriented sensor described above, along with a processor and display. In this system, the angular orientation information provided by the sensor, e.g., data from the accelerometer, may be transmitted to a device that processes the information to display the angles at which the tool is oriented. As such, the surgeon or other user may readily see the angular orientation of the tool or instrument so that it may be positioned at the desired trajectory. For example, the angular information provided by the sensor may be processed so that the axial angle and sagittal angle of the instrument with respect to a horizontal or lateral plane may be displayed.

The angular orientation information provided by the sensor is generally with respect to a vertical plumb line. But because the sensor axis and tool axis are maintained perpendicular to each other, the information from the sensor also indicates the angles at which the tool is positioned with respect to a horizontal or lateral plane.

Another aspect of the invention involves an app that may be downloaded onto a tablet or other device that may include a display. The tablet or other device may be paired with the sensor via Bluetooth or other communication protocols. The downloaded app may process the angular orientation information received from the sensor and display the angles at which the tool or instrument is oriented. Alternatively, processing may occur in the sensor chip or assembly and then be transmitted to the device providing the display.

Another aspect of the invention regards the sensor being oriented at an angle other than perpendicular to the tool. For example, the sensor may be positioned at 45 degrees, 60 degrees or any other desired angle in relation to the shaft of a tool, or the sensor may be positioned at any known, fixed angle in relation to the tool or other device. The angular orientation information provided by the sensor may be processed to display pertinent angles.

### BRIEF DESCRIPTION OF THE DRAWINGS

Various objects, features and attendant advantages of the present invention will become fully appreciated when considered in conjunction with the accompanying drawings, in which like reference characters designate the same or similar parts throughout the several views, and wherein:

FIG. 1A shows a side view of a mechanism that may accommodate a surgical instrument and that may accommodate a sensor.

FIG. 1B shows a top view of the mechanism in FIG. 1A.

FIG. 1C is a perspective view of a mount shown in FIGS. 1A and 1B.

FIG. 2 shows a side view of an awl that may accommodate a sensor.

FIG. 3 shows a side view of a screwdriver that may accommodate a sensor.

FIG. 4A shows a side view of a cannulated needle that may accommodate a sensor.

FIG. 4B shows a top view of a cannulated needle that may accommodate a sensor.

FIG. 5 shows a system including a tool that is intended for lateral use and that includes a vertically oriented sensor, along with a display.

FIG. 6 shows a sensor that may be attached to an item that is desired to be installed or to reside at a particular angle as measured from horizontal.

The figures of U.S. Pat. No. 11,484,381 are also referenced herein, and are incorporated by reference as if fully set forth herein.

### DETAILED DESCRIPTION OF THE PREFERRED EMBODIMENTS

In general, the system according to exemplary embodiments hereof provides, devices, tools, instrumentation, software and methods to assist in the proper alignment of devices. The devices to be aligned may include hand tools and/or instruments of any kind, for example, including but not limited to: surgical instruments, hand drills, screw drivers, awls, probes, taps, saws, files, plyers, tweezers, scalpels, hypodermic needles, cannulated drill guides, jamshidi needles, cannulated needles and other types of hand held devices, non-hand held devices and tools. It will be understood by a person of ordinary skill in the art, upon reading this specification, that the system and method may be used with any type of device that may benefit from being aligned in one, two or three dimensions, and that the scope of the system and method is not limited in any way by the types of devices that it may be used with.

For the purpose of this specification and for demonstration, the system and method of the current invention is described in relation to its use with surgical instruments (e.g., instruments used for orthopedic surgery). However, it is appreciated that the system and method of the current invention may be applied to and used with any type of tool, instrument or device that may benefit from it, i.e., where the tool, instrument or device is intended to be used at a desired trajectory or angular orientation that is generally more lateral or horizontal, than vertical. This may include other tools, instruments or devices for other medical applications such as dentistry, as well as for industrial applications, machine applications and other non-surgical applications.

The current invention may be particularly applicable to tools, instruments or devices that have a shaft, but the current invention is not limited to such items. To this end, the current invention may be used with items that are not tools used to act on something, e.g., an awl acting on a pedicle. For example, a component in machinery may need to be positioned at a precise angle to meet assembly tolerances of that machinery. Where the angle of that component is to be measured from a horizontal plane, the current invention may display the lateral angles at which the component resides. That is, a sensor may be attached to a surface of the component at a known angle, e.g., 90 degrees.

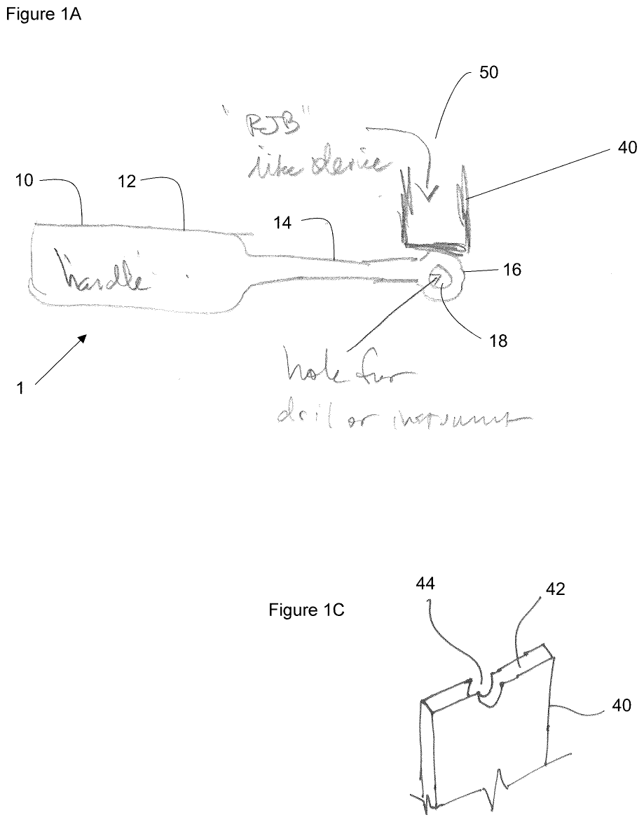

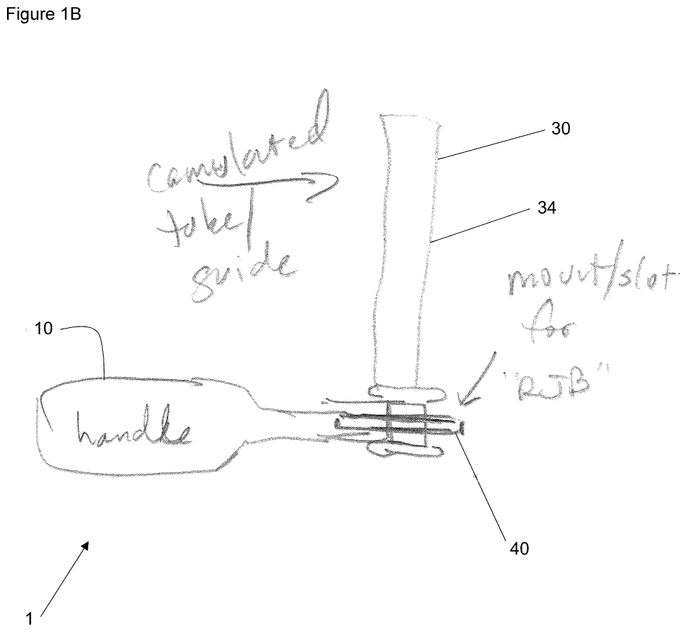

Referring to FIGS. 1A and 1B, an embodiment of the lateral alignment system 1 of the current invention is now described. As shown, system 1 may include handle or tool 10, surgical instrument (or other type of non-surgical instrument or tool) 30 and angular alignment sensor 50. Sensor 50 may be as described as measurement assembly 100 in U.S. Pat. No. 11,484,381, incorporated by reference herein.

FIG. 1A is a side view showing handle 10 without yet having received tool 30, while the top view of FIG. 1B shows handle 10 having received tool 30. Handle 10 may include gripping, distal or handle section 12 which may be grasped and manipulated by a surgeon or other user of system 1. Shaft section 14 may extend proximally from gripping section 12 to proximate or mounting section 16. Handle section 12 may be offset from shaft section 14 at an angle that may be suitable to assist the surgeon during particular surgical procedures.

Mounting section 16 may include hole or bore 18 to receive a surgical instrument or other tool or device 30, as shown in FIG. 1B . As shown in FIG. 1B, tool 30 may comprise a cannulated tube or guide having shaft 34. Cannulated tube 30 may be positioned laterally to accommodate another instrument. Alternatively, surgical instrument 30 may comprise an awl, drill or other type of instrument desired by the surgeon. Where shaft 34 of surgical instrument or other tool or device 30 has a circular cross-section, hole 18 may have a diameter that may correspond to the diameter of the instrument shaft 34. Alternatively, hole 18 may be a square or other shape that corresponds to the shape of tool 30 or its shaft 34.

The engagement between tool 10 and instrument 34 is preferably secure so that the trajectory of instrument 34 is precise and as intended by the surgeon. To this end, a secure friction fit may exist between hole 18 and instrument 34. Alternatively, a locking device may be used to secure instrument 34 to mounting section 16. A quick release mechanism may also be used so that different instruments 34 may be used with tool 10 where desired for a particular surgical procedure.

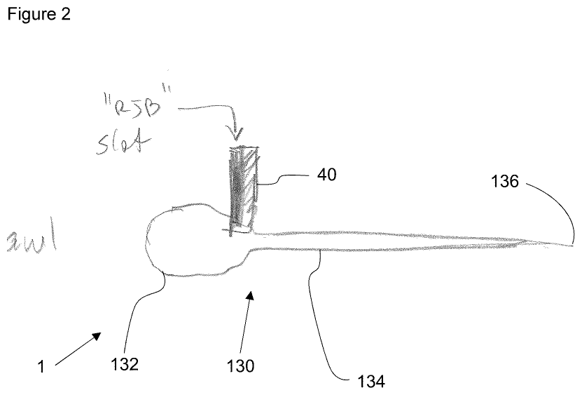

Handle 10 may also include a mount or bracket 40 to receive an alignment sensor 50, such as an RJB™ sensor chip distributed by Ruthless Spine, LLC. Mount 40 may be attached to handle 10 at its proximal end, as shown in FIGS. 1A and 1B , but may alternatively be attached at different locations of handle 10, such as shaft 14. Mount 40 may be attached on top of handle 10, as shown in FIGS. 1 and 2, or on the underside of handle 10. It is preferred that mount 40 be located so as to avoid interfering with the surgeon's or other user's use of system 1.

Mount or bracket 40 may be attached to handle 10 with appropriate fasteners, or may be 3D printed or molded as part of handle 10. Alternatively, mount or bracket 40 may clip on to handle 10, e.g., clip on to shaft 14, and may be removed later on. In this embodiment, the surgeon or other user may position mount 40 (and thus alignment sensor 50) at a desired location which may vary according to the use of system 1.

As shown in more detail in FIG. 1C, mount or bracket 40 may generally include slot 42 that is dimensioned so that alignment sensor 50 fits snugly therein. Slot 42 may include gaps 44 that may allow sensor 50 to be pulled out of slot 42. Bracket or mount 40 may be formed so that its inferior and lateral surfaces comprise a solid slot that folds over the superior surface of alignment sensor 50 slightly, i.e., enough to secure alignment sensor 50 in place, but still allowing access by a finder to slide sensor 50 back out of slot 42.

In another embodiment, sensor 50 may be formed integrally with mount or bracket 40 so that it is not removable therefrom. Sensor 50 may also be formed integrally with handle 10 so that it is all one piece, e.g., where tool or handle 10 and sensor 50 are intended for only a single use.

Regardless of whether sensor chip 50 is to be removable from mount 40 or not, system 1 preferably maintains a perpendicular relationship between the axis of the sensor chip 50 and the axis of the tool 30 after it is received by handle 10. Where mount 40 is clipped on to shaft 14, the interior surfaces of the clip may include protrusions that may engage depressions in the surface of shaft 14. Alternatively, the interior surfaces of the clip may include depressions and the surface of shaft 14 may include protrusions. These protrusions and depressions may be located to ensure that the sensor 50 is oriented vertically or perpendicularly from the shaft of handle 10 and/or longitudinal axis of instrument 30.

With the axis of instrument 30 and the axis of sensor 50 being perpendicular to each other, the angle between the sensor axis and the vertical plumb line will be equal to the angle between the laterally positioned instrument and a horizontal plane. As such, the current invention allows sensors 50 that are programmed or otherwise designed to provide angular orientation information with respect to a vertical plumb line, e.g., sensors 50 having an accelerometer that is positioned along the direction of gravity, to be used to provide trajectory information for laterally positioned tools, i.e., the trajectory of laterally positioned tools with respect to a horizontal plane.

This advantageously avoids having to reprogram sensors that are intended to work with a vertical plumb line to work with a horizontal plumb line. This also avoids the situation where a sensor may otherwise need to include two accelerometers, with one oriented along a vertical plumb line and the other oriented with respect to horizontal. This also avoids the situation where the sensor may need to be otherwise manipulated to provide angular orientation information with respect to horizontal.

The current invention also advantageously allows processors and displays that are designed to work with such sensors designed or programmed to work with a vertical plumb line, to be used for lateral trajectories without redesign efforts. For example, the processor will not need to be programmed to convert angular measurement information from vertically oriented information to horizontally oriented information. Furthermore, the displaying of angles 502, 504 with respect to horizontal, may generally be the same as the angles that would be displayed with respect to vertical. It may be preferred that when the display of angles 502, 504 reflect lateral angles, the display may be a different color than would be displayed for angles 502, 504 reflecting vertical angles. Alternatively, some other indicia may be displayed by device 500 to indicate that the displayed angles 502, 504 are with respect to horizontal.

Referring again to FIG. 1B, when tool 30 is inserted into handle 10, tool 30 may generally be laterally positioned, or in generally at or near a horizontal plane. This preferably facilitates a surgeon performing surgical procedures that are desired to be performed laterally. The tool 30 shown in FIG. 1B may be a cannulated tube or guide that is laterally positioned and that may receive another surgical instrument so that it too is oriented laterally.

When tool 30 is oriented laterally, mount 40 serves to position sensor chip 50 in a vertical direction. As such, the tool 30 and sensor 50 may be kept perpendicular to each other. In this manner, sensor chip 50, that may be programmed or otherwise designed to provide angular alignment information with respect to a vertical plumb line, may still be used even though the tool 30 is oriented laterally. In other words, the angular deflection of the tool 30 from horizontal will be the same as the angular deflection of the sensor 50 from the vertical plumb line.

System 1 may provide angular orientation information of the laterally positioned tool 30 with respect to a horizontal plane, e.g., the floor, as follows. As noted above, sensor 50 may be oriented perpendicularly from the axis of tool 30. So when the tool 30 is on a horizontal plane, i.e., the shaft of tool 30 is on a horizontal plane, sensor 50 will be vertical, or at zero degrees from a vertical plumb line.

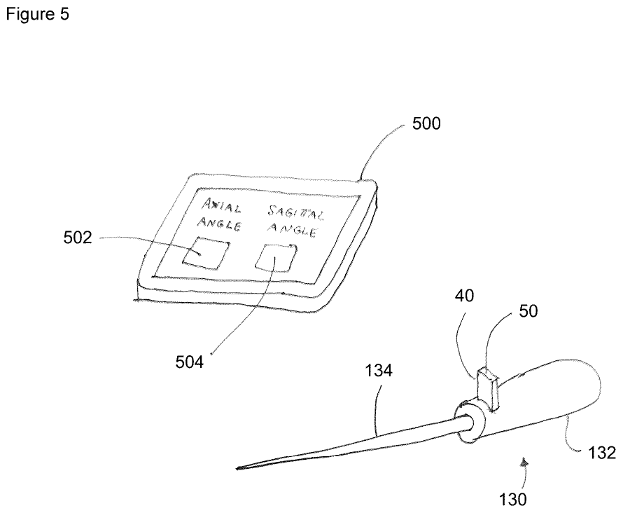

As noted in U.S. Pat. No. 11,484,381, which is incorporated by reference as if fully set forth herein, and as shown in FIG. 5 herein, sensor 50 may be in communication with device 500 that may include a display that displays the lateral angular orientation of instrument 30. Device 500 may be a tablet, smartphone, laptop or other types of devices. Sensor 50 and device 500 may communicate through a Bluetooth protocol or other types of communication.

Device 500 may be programmed, e.g., an app may be downloaded on device 500, to receive angular orientation information from sensor 50, and to process this information to calculate and display trajectory values, e.g., axial angle 502 and sagittal angle 504 with respect to horizontal. Alternatively, angular orientation information may be processed by sensor 50 and sent to device 500 for display trajectory values 502, 504.

Device 500 may display the amount of angular deflection of whatever tool or instrument is used from horizontal, based on the amount of angular deflection of sensor 50 from vertical. So where the tool or instrument is positioned horizontally, the display may indicate zero degrees deflection because the sensor will be deflected zero degrees from the vertical plumb line.

Another embodiment of system 1 regarding an awl 130 is now described with reference to FIGS. 2 and 5. In this embodiment, system 1 need not have a separate handle as shown in FIGS. 1A and 1B. Instead, awl 130, including sensor 50, may comprise system 1. In this embodiment, awl 130 may include a handle or gripping section 132 for the surgeon or other user to manipulate. Awl 130 may also include shaft 134 that may conclude proximally with a pointed end 136.

Awl 130 may include mount 40 to receive sensor 50 as described above. Mount 40 is preferably configured so that it positions sensor 50 at a 90 degree angle from the axis of shaft 134. In this manner, angular information provided by sensor 50 with respect to a vertical plumb line may reflect the angular orientation of the shaft 134 from horizontal. Details described above in connection with the embodiment of FIGS. 1A and 1B, such as the type of mount 40, its location and the manner in which it engages sensor 50, also apply to this embodiment. Awl 130 with its sensor 50 may operate with device 500 as discussed above.

As shown in FIGS. 2 and 5, mount 40 is preferably located at or near proximal end of handle 132 to avoid interfering with the surgeon's grasping of handle 132. And as shown more clearly in FIG. 5, it is also preferred that the long dimension of mount 40 is oriented transverse to shaft 134. In this configuration, mount 40 does not extend axially along handle 132.

Furthermore, this configuration of mount 40 and sensor 50 generally represents the manner in which measurement assembly 100 is oriented with respect to gravity as described in U.S. Pat. No. 11,484,381, incorporated by reference herein. As described in the '381 patent, the RJB™ sensor, or measurement sensor 100, may be positioned along the axis of a surgical instrument, and is generally used with respect to gravity, i.e., a vertical plumb line. As shown in FIGS. 2 and 5, mount 40 and sensor 50 are configured similarly to the RJB™ sensor with respect to gravity, but are also positioned perpendicularly from shaft 134. As such, an RJB™ sensor, or other sensor designed or programmed to work with reference to gravity, may still be used to provide angular measurement information with respect to horizontal.

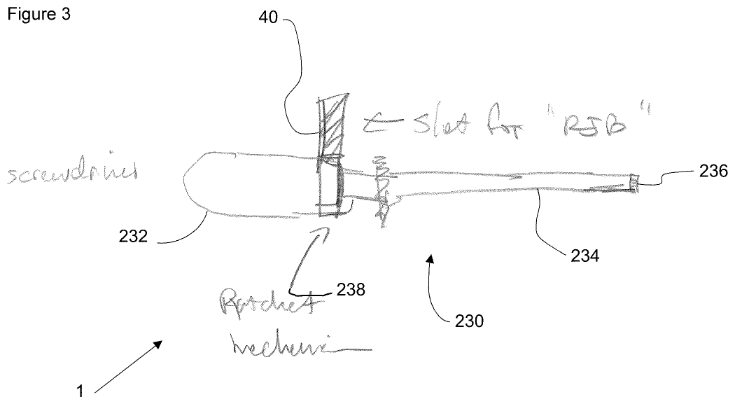

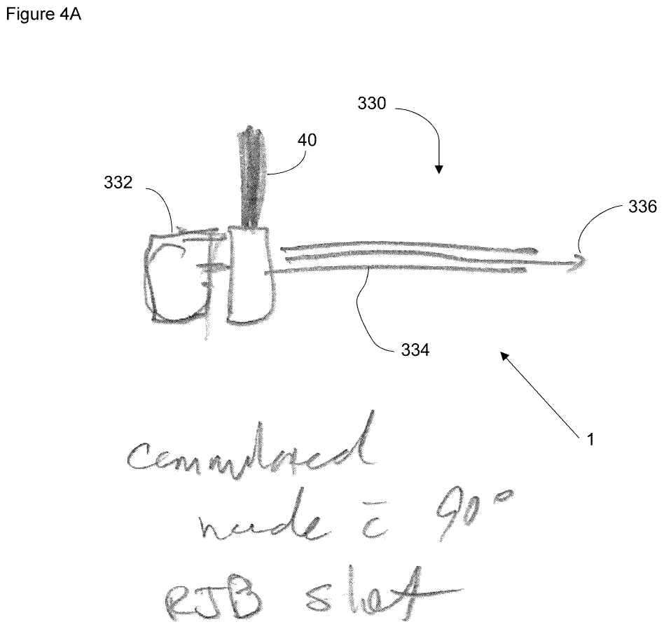

Another embodiment of system 1 regarding a screwdriver 230 is now described with reference to FIG. 3. In this embodiment, system 1 need not have a separate handle as shown in FIGS. 1A and 1B. Instead, screwdriver 230, including sensor 50, may comprise system 1. In this embodiment, screwdriver 230 may include a handle or gripping section 232 for the surgeon or other user to manipulate. Screwdriver 230 may also include shaft 234 that may conclude proximally with a desired screwdriver configuration 236, e.g., Philips or flat configuration. Screwdriver 230 may also include a ratchet mechanism 238 to allow the surgeon or other user to repeatedly rotate shaft 234 so that a pedicle screw may be implanted or other item is screwed into a secure position.

Screwdriver 230 may include mount 40 to receive sensor 50 as described above. Mount 40 is preferably configured so that it positions sensor 50 at a 90 degree angle from the axis of shaft 234. In this manner, angular information provided by sensor 50 with respect to a vertical plumb line may reflect the angular orientation of the shaft 134 from horizontal. Details described above in connection with the embodiment of FIGS. 1A and 1B, such as the type of mount 40, its location and the manner in which it engages sensor 50, also apply to this embodiment. Screwdriver 230 with its sensor 50 may operate with device 500 as discussed above.

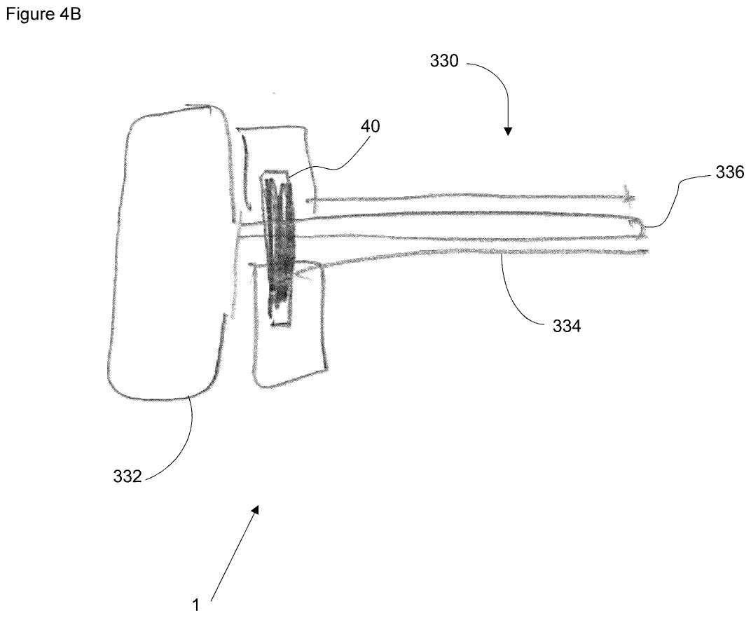

Another embodiment of system 1 regarding a jamshidi needle 330 is now described with reference to FIGS. 4A and 4B. In this embodiment, system 1 need not have a separate handle as shown in FIGS. 1A and 1B. Instead, jamshidi needle 330, with sensor 50, may comprise system 1. In this embodiment, jamshidi needle 330 may include a handle or gripping section 332 for the surgeon or other user to manipulate. Jamshidi needle 330 may also include shaft 334 that may conclude proximally with a desired cannula and needle 336.

Jamshidi needle 330 may include mount 40 to receive sensor 50 as described above. Mount 40 is preferably configured so that it positions sensor 50 at a 90 degree angle from the axis of shaft 334. In this manner, angular information provided by sensor 50 with respect to a vertical plumb line may reflect the angular orientation of the shaft 334 from horizontal. Details described above in connection with the embodiment of FIGS. 1A and 1B, such as the type of mount 40, its location and the manner in which it engages sensor 50, also apply to this embodiment. Jamshidi needle 330 with its sensor 50 may operate with device 500 as discussed above.

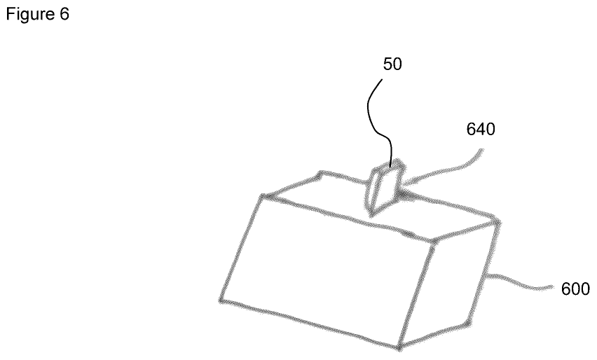

Another application of the current invention is now described with reference to FIG. 6. This embodiment refers to accurately positioning an item 600 at desired angles. For example, item 600 may comprise a component that is installed in a machine, e.g., an engine or other mechanical device. With such machines, components must oftentimes be precisely installed so that the machine may operate properly. For example, mechanical parts in an engine or other machine where the parts move, must be precisely installed so that surfaces are not worn down, etc.

In this embodiment, item 600 represents such a component that is preferably installed as part of an assembly in a precise fashion including that it must be positioned at an angle relative to horizontal. In this embodiment, mount 640 may contain sensor 50. Mount 640 may be removably attached to a surface of component 600 that is known to be horizontal. In this manner, sensor 50 may be positioned perpendicular to this surface and can provide angular orientation information so that component 600 is installed at a desired angle relative to horizontal.

Other aspects of the current invention are now described. The foregoing description focuses on the situation where sensor 50 is perpendicular to an instrument so that the angle between sensor 50 and a vertical plumb line represents the angle between the instrument and horizontal. However, sensor 50 may be maintained at angles other than 90 degrees relative to the laterally positioned instrument. For example, sensor 50 could be maintained at 45 degrees or 60 degrees, or at some other known angle relative to the shaft of the instrument. Such non-perpendicular arrangements may be useful where the user is concerned with the angular orientation of an instrument relative to a plane that is not horizontal. In this embodiment, some reprogramming of existing vertically oriented sensors, or the app that processes the angular orientation information received from the sensor, may be necessary.

The use of the current invention during surgical procedures is now further described. As noted earlier, there are increasing types of spinal surgery performed in the lateral position where the patient is positioned on his or her side, thereby positioning the spine laterally. Where the surgery is to involve the surgical instruments engaging the patient from above, i.e., generally vertically, sensors such as the RJB™ sensor programmed to work with gravity, may be used to provide angular measurements with respect to vertical.

However, where the patient is lying on his or her side, the pedicles are oriented laterally, i.e., 90 degrees from vertical. So where pedicle screws are to be implanted while the patient is lying on his or her side, the surgical instruments used would generally be engaging the patient horizontally. The current invention addresses this situation by involving a sensor 50 that may be programmed to work with gravity, but may still provide angular information with respect to horizontal because it is oriented perpendicularly from the shaft of the laterally positioned instrument.

Where such surgery is to include procedures where the surgical instruments enter the patient laterally, as well as from the back, the current invention contemplates providing a set of instruments including an instrument having an angular measurement sensor aligned with its axis, such as described in the '381 patent, and another instrument having an angular measurement sensor aligned perpendicularly to its axis.

Laterally performed spinal surgery procedures, where the patient is in a prone position, are being developed. In this situation, where the surgical instrument enters the patient laterally, the axis of the surgical instrument would again generally perpendicular or at a significant angle from a vertical plumb line. The current invention again addresses this situation by involving a sensor 50 that may be programmed to work with gravity, but may still provide angular information with respect to horizontal because it is oriented perpendicularly from the shaft of the laterally positioned instrument.

The current invention may also be used in Single Position Lumbar Surgery (SPLS), which has been shown to decrease anesthesia/surgical time, because the patient need not be positioned at both lateral and prone positions to complete the surgical procedures at issue. However, SPLS may be technically challenging due to the position of the patient and the loss of proprioceptive clues when emplacing pedicle screws.

However, spinal navigation may render those technical challenges moot with the use of the current invention. To this end, an RJB™ sensor, or other sensor 50 designed or programmed to work relative to gravity, may be positioned in mount 40 so as to be perpendicular to the surgical instrument(s) being used when the instruments are being used laterally. This preferably makes intuitive sense to a surgeon trained to thinking about the spine in a prone position when operating. In other words, the current invention may convert the 3D anatomy in the surgeon's mind to a configuration with which the surgeon is familiar without the disadvantages of traditional spinal navigation (TSN). That is, there is little or no learning curve for the surgeon and the surgeon may still place the LLIF cage using traditional techniques for placement that had existed before TSN was available.

As noted above, U.S. Pat. No. 11,484,381 is incorporated by reference as if fully set forth herein. This patent provides detail regarding the components of the systems described herein and their use.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. An instrument guide, comprising: a mount that is configured to receive an angular measurement sensor which is programmed to work in the direction of gravity and that is configured to position the angular measurement sensor perpendicular to an axis of an instrument.

2. The instrument guide of claim 1, wherein the mount includes a slot to receive the angular measurement sensor.

3. The instrument guide of claim 1, wherein the mount is configured to be attached to the axis of the instrument, thereby positioning the angular sensor guide at an angle perpendicular to the axis of the instrument.

4. The instrument guide of claim 3, wherein the angular measurements of the instrument relative to horizontal are provided by the angular measurement sensor providing angular measurements with respect to gravity.

5. A surgical instrument for use in a lateral position, comprising:
a shaft that is positioned at or near horizontal during use;
a mount attached to the shaft, and
an angular sensor that is received by the mount and that is programmed to provide angular measurements with respect to gravity.
